(19) United States
(12) Patent Application Publication
(10) Pub. No.: US 2021/0145761 A1
(43) Pub. Date: May 20, 2021

(54) TRANSDERMAL DELIVERY SYSTEM CONTAINING A DOPAMINE AGONIST

(71) Applicant: AMW GMBH, Warngau (DE)

(72) Inventors: Stefanie RODLER, Valley (DE); Sonja SEITZ, Hausham (DE)

(73) Assignee: AMW GMBH, Warngau (DE)

(21) Appl. No.: 16/757,290

(22) PCT Filed: Oct. 19, 2018
(86) PCT No.: PCT/EP2018/078731

§ 371 Date: Apr. 17, 2020

(30) Foreign Application Priority Data
Oct. 20, 2017 (DE) 10 2017 124 627.4

(51) Int. Cl. A61K 9/70; A61K 31/381; A61K 47/32; A61K 47/14; A61K 47/10; B65D 81/26

(52) U.S. Cl. CPC A61K 9/7084; A61K 31/381; A61K 47/32; B65D 81/266; A61K 47/10; A61K 9/7092; A61K 47/14

(57) ABSTRACT

The present invention relates to a transdermal delivery system (TDS) comprising at least one dopamine agonist, wherein the at least one dopamine agonist is present in a matrix containing at least one polymer from the class of polybutylene. The present invention also relates to a method for producing the TDS according to the invention and to the use of the TDS according to the invention.

The present invention relates to a transdermal delivery system (TDS) comprising at least one dopamine agonist, wherein the at least one dopamine agonist is present in a matrix containing at least one polymer from the class of polybutylenes. The present invention also relates to a method for producing the TDS according to the invention and to the use of the TDS according to the invention.

Dopamine agonists are drugs that can cause effects in the organism similar to the body's own dopamine neurotransmitter, primarily by stimulating dopamine receptors. Dopamine agonists are divided structurally into the class of ergoline and non-ergoline dopamine agonists or, in accordance with their binding affinity, into the class of D_{1/5} or D_{2/314} agonists. They are used for the treatment of Parkinson's disease, amenorrhoea, acromegaly, as emetics, for weaning during the breastfeeding period, and as impotence drugs.

Previously, the active ingredient L-DOPA (L-3,4-Dihydroxyphenylalanine), a natural precursor of dopamine, was the gold standard for the treatment of diseases in which an increase in the dopamine level is advantageous. However, side-effects occur in the case of therapy with L-DOPA, for example dyskinesia.

Dopamine agonists are able to completely replace the active ingredient L-DOPA in earlier stages of an illness and may alleviate the side-effects of L-DOPA by adjunct therapy in later stages of an illness. However, dopamine agonists may themselves cause strong side-effects, the cause of which may be attributed to the pulsatile administration of the drug (Samuel et al., Mov. Disord., 2015), whereby the active ingredient concentration in the blood plasma may be subject to strong fluctuations. Most dopamine agonists when administered orally are therefore at least partially metabolised or retained during the first passage through the intestinal tract and the liver (first-pass effect). Thus, only a fraction of the orally administered active ingredient in its active form reaches the target site. Furthermore, most dopamine agonists have short plasma half-life periods, and therefore initially high active ingredient concentrations in the blood plasma may decrease already after a short time.

For the above reasons, it is important to develop a safe and effective pharmaceutical delivery form, with the aid of which controlled amounts of the drug can be administered. Transdermal delivery systems constitute an expedient alternative for the administration of dopamine agonists, since active ingredients, as a result of this kind of administration, can be made available in controlled fashion and within the particular therapeutic dose. Transdermal delivery systems (syn. transdermal therapeutic systems, or TTS) are generally flexible, adhesive preparations for the controlled delivery of one or more active ingredients for application to the skin. The active ingredient may be resorbed from the TDS through the skin over a period of from approximately a day to approximately seven days, and may pass directly into the bloodstream without significant metabolisation. Therapy often occurs over months or years.

A precondition for a successful systemic provision of the active ingredient with the aid of transdermal delivery systems is the highest possible active ingredient delivery rate of the TDS. The active ingredient delivery rate is substantially dependent on the concentration of the active ingredient in the matrix of the transdermal administration system and the physicochemical properties of the active ingredient.

In order to attain an increased active ingredient delivery rate, active ingredient concentrations exceeding the saturation solubility are often dissolved in the active ingredient-containing matrix of the TDS. Due to the high concentration of the active ingredient in the active ingredient matrix, however, such systems oversaturated with active ingredient often tend towards crystallisation during storage (Stefano et al. 1997, Variankaval et al. 1999, Lipp et al. 1999, Kim & Choi). A crystallisation of the active ingredient, however, reduces the thermodynamic activity thereof, and consequently the active ingredient delivery rate, and is therefore usually delimited by the addition of solubility enhancers.

It is known, however, that solubility enhancers may load the organism undesirably, for example by allergic reactions and/or skin irritations. The addition of solubility enhancers may also mean that the adhesive force of the transdermal delivery system decreases.

The transdermal delivery systems described in the prior art, however, have either a complex structure and require a multitude of different excipients in order to produce TDS having high active ingredient delivery rates, or lack the necessary therapeutic active ingredient dose by a large margin.

WO 02/015903 A2 discloses a rotigotine-containing depot form with which, following application in a patient, a therapeutically relevant plasma level can be achieved over at least 24 hours.

The object of the present invention is therefore to provide a transdermal delivery system having a content of at least one dopamine agonist, wherein the TDS comprises an active ingredient-containing matrix containing at least one polymer from the class of polybutylene and demonstrating an efficient active ingredient release rate and good storage stability.

This object is achieved in accordance with the invention by a TDS having an active ingredient-containing matrix which, according to claim 1, contains at least one polymer from the class of polybutylenes, and by a method for producing the TDS according to the invention according to claim 11. The object is also achieved by a composition according to the invention for use according to claim 14.

The present invention therefore relates to a transdermal delivery system comprising
(i) at least one dopamine agonist in at least one matrix,
(ii) an active ingredient-impermeable rear layer, and
(iii) an optional application-side protective film,
   wherein the matrix containing the at least one dopamine agonist comprises at least one polymer from the class of polybutylenes, in particular polyisobutylenes.

Transdermal delivery systems according to the invention as per claim 1 advantageously have a good storage stability, in particular in respect of a potential crystallisation, and at the same time have a controlled and good active ingredient delivery rate.

In its simplest embodiment the transdermal therapeutic systems comprise a rear layer and at least one dopamine agonist in at least one matrix containing at least one polymer from the class of polybutylenes, in particular polyisobutylenes. The matrix layer is arranged horizontally in the TDS and is used to attach the TTS to the skin.

The side of the TDS generally intended for attachment to the skin or coming into contact with the skin is referred to as the application side. The application side, to this end, may be adhesive over the entire area, for example in that the dopamine agonist-containing matrix itself is adhesive.

A TDS according to the invention may optionally contain a protective layer which is applied to the dopamine agonist-containing matrix and is removed before application of the TTS.

As mentioned above, the at least one dopamine agonist is advantageously contained in a matrix which contains at least one polymer from the class of polybutylenes. Hereinafter, the expression “dopamine agonist-containing matrix containing at least one polymer from the class of polybutylenes” is equivalent to a matrix containing at least one dopamine agonist and based on polybutylene. The term “polybutylene (PB)” is equivalent here to polybutene. In particular, a polybutylene may comprise a polymer which at least partially contains one or more isobutylenes or polyisobutylenes (PIB) and/or is fully replaced by one or more polyisobutylene(s).

In accordance with the invention the at least one dopamine agonist is contained in a matrix based on polybutylene, in particular based on polyisobutylene. A matrix based on polybutylene, in particular based on polyisobutylene, is understood to mean a matrix layer which, based on all substances contained in the matrix, in particular active ingredients and optionally contained excipients, has a total content of polybutylene or polyisobutylene which is preferably at least 25% by weight, particularly preferably at least 35% by weight, in particular at least 45% by weight, and in particular preferably at least 50% by weight. In principle, the preferred proportion of adhesive may be up to approximately 95% by weight, however it is particularly preferred if the proportion of the adhesive is no more than approximately 80% by weight. The total content of polybutylene here comprises the content of all polybutylene polymers or polyisobutylene polymers in the dopamine agonist-containing matrix.

The transdermal therapeutic systems according to the invention are suitable for the application of, in principle, all dopamine agonists, such as dihydrexidine, cabergoline, dihydroergocryptine, terguride, dinapsoline, dinoxyline, etilevodopa, melevodopa, ibopamine, pergolide, lisuride, quinagolide, rotigotine, bromocriptine, apomorphine, pramipexole, ropinirole, propylnorapomorphine, sumanirole, piribedile and roxindole.

The transdermal therapeutic system according to the invention delivers the dopamine agonists from the active ingredient-containing matrix based on polybutylene to the skin or into the skin, wherein a significant part of the active ingredient is taken up systemically.

The present invention thus also relates to the medical and/or veterinary use of the patches according to the invention (the TTS will also be referred to hereinafter as a patch) for delivering dopamine agonists, in particular D₂ agonists, to or through the skin of a human or animal body.

The present invention also relates to a method for producing a TDS according to the invention, wherein the method comprises the following steps:
(i) providing at least one dopamine agonist-containing matrix containing at least one polymer from the class of polybutylenes, in particular polyisobutylenes,
(ii) optionally applying the at least one dopamine agonist-containing matrix to a film in order to obtain a laminate comprising a dopamine agonist-containing matrix,
(iii) optionally drying the laminate comprising the at least one dopamine agonist-containing matrix,
(iv) optionally punching out transdermal delivery systems in order to obtain planar active ingredient cores,
(v) optionally packaging transdermal delivery systems.

The term “providing” in this case is understood to mean both a production locally and a delivery of a dopamine agonist-containing matrix comprising at least one polymer from the class of polybutylenes, in particular polyisobutylenes. Here, a dopamine agonist-containing matrix which comprises at least one polymer from the class of polybutylenes may already be provided with a protective film covering the application side of the dopamine agonist-containing matrix, which polymer remains on the application side during the further course of the production process or optionally may be replaced by an alternative protective film in one or more production steps.

In accordance with a preferred method the at least one dopamine agonist-containing matrix may be applied to a film so as to obtain a laminate. Here, a laminate may be understood to be a material or a product which consists of two or more layers bonded flat to one another. These layers may consist of the same or different materials, but preferably consist of different materials.

Lastly, the present invention comprises a transdermal therapeutic system which is obtainable by a method as described above.

Further particularly advantageous embodiments and developments of the invention result from the dependent claims and the following description, wherein the claims of a specific category may also be developed in accordance with the dependent claims of another category, and features of various exemplary embodiments may be combined to form new exemplary embodiments.

In accordance with a preferred embodiment, the TTS in the at least one matrix comprising a polymer from the class of polybutylenes, in particular polyisobutylenes, contains a dopamine agonist from the class of D₂ agonists or the ergot alkaloids or derivatives thereof, particularly preferably from the class of D₂ agonists, in particular from rotigotine, pramipexole and/or ropinirole, in particular preferably from rotigotine.

The at least one dopamine agonist may be contained in the matrix in different forms, depending on which form gives the optimal delivery property of the active ingredient from the TDS or the matrix. Generally, dopamine agonists may be present in the form of the free base or acid, esters or other pharmacologically acceptable derivatives or as components of molecular complexes. The at least one dopamine agonist, in particular rotigotine, however, is preferably present in the form of the free base.

The amount of dopamine agonists, preferably of the D₂ agonist, in particular of rotigotine, in the system may advantageously vary from at least approximately 0.3% by weight, preferably from at least 3% by weight, particularly preferably from at least approximately 4.5% by weight, in particular from at least approximately 7% by weight. At most, however, the content of the dopamine agonists, preferably of the D₂ agonist, in particular of rotigotine, is preferably up to approximately 50% by weight, particularly preferably up to approximately 15% by weight, in particular up to approximately 12.5% by weight, in particular preferably up to approximately 9% by weight.

The absolute active ingredient amount contained in the patch generally determines the time period over which a continuous supply of the active ingredient into or to the organism is maintained. Thus, the highest possible loading of the matrix based on polybutylene with dopamine agonists is then desirable if the application time of a patch is long, i.e. a number of days to a week. A transdermal therapeutic system according to the invention, however, is preferably used for an application period of from one to four days, in particular for an application duration of from one to three days, in particular preferably of from one to two days.

In principle, a suitable weight per unit area of the dopamine agonist-containing matrix based on polybutylene moves within a range that is usual for transdermal therapeutic systems. Insofar as a preferred transdermal therapeutic system is intended for an application period of approximately one day, a preferred weight per unit area of at least one dopamine agonist-containing matrix based on polybutylene is at least approximately 10 mg/10 cm², particularly preferably at least approximately 20 mg/10 cm², in particular at least approximately 30 mg/10 cm², in particular preferably at least approximately 40 mg/10 cm². At most, a preferred weight per unit area is up to approximately 100 mg/10 cm², particularly preferably up to approximately 80 mg/10 cm², in particular up to approximately 75 mg/10 cm², in particular preferably up to approximately 70 mg/10 cm².

If an advantageous transdermal therapeutic system is to be used for more than a day, a preferred weight per unit area may also lie above approximately 100 mg/10 cm². For example, a preferred transdermal therapeutic system with an application time of approximately two days may thus have a weight per unit area of up to approximately 400 mg/10 cm², particularly preferably of up to approximately 300 mg/10 cm², in particular of up to approximately 200 mg/10 cm². An advantageous transdermal therapeutic system with a wearing time of approximately three days preferably has a weight per unit area of at least approximately 150 mg/10 cm², particularly preferably of at least approximately 200 mg/10 cm², in particular of at least approximately 300 mg/10 cm². At most, a transdermal therapeutic system with a wearing time of approximately three days has a weight per unit area of preferably up to approximately 500 mg/10 cm², particularly preferably of up to approximately 400 mg/10 cm².

The described transdermal therapeutic systems may in principle comprise both one or more matrix layer(s) which in each case contain at least one polymer from the class of polybutylenes or polyisobutylenes.

Insofar as transdermal therapeutic systems consist of two or more matrices based on polybutylene or polyisobutylene, merely one matrix layer may comprise at least one dopamine agonist, or the at least one dopamine agonist active ingredient may also be contained in further matrix layers. Furthermore, said matrices based on polybutylene may have a different composition from one another. In principle, all combinations of dopamine agonists with affinity to all classes of dopamine receptors are possible.

For example, a first matrix layer of the TDS may contain a dopamine D₂ agonist, and a second or further matrix layer of the TDS may contain a dopamine D₁ agonist. Examples of dopamine D₁ agonists are dihydrexidine, cabergoline, dihydroergocryptine, terguride, dinapsoline, dinoxyline, etilevodopa, melevodopa, ibopamine, pergolide and lisuride. Examples of dopamine D₂ agonist are quinagolide, rotigotine, bromocriptine, cabergoline, apomorphine, pramipexole, ropinirole, propylnorapomorphine, sumanirole, pergolide, piribedile, dihydroergocryptine and lisuride. Of course, combinations of dopamine agonists with affinity to the further dopamine receptor classes are also possible. The class of dopamine D₃ agonists thus comprises, for example, pramipexole, ropinirole, propylnorapomorphine, pergolide, piribedile and lisuride. Representatives of dopamine D₄ agonist are, for example roxindole and lisuride. Examples of dopamine D₅ agonists are dihydrexidine and also lisuride. In accordance with a particularly preferred embodiment, however, the TDS comprises merely dopamine agonists with affinity to the same class determined by the receptor affinity, in particular to dopamine D₂ receptors, in particular preferably rotigotine.

Lastly, an application-side matrix may be both free from active ingredient or also may comprise one or more dopamine agonists, however, an application-side matrix preferably comprises a dopamine agonist.

The at least one dopamine agonist-containing matrix based on polybutylene or polyisobutylene or also a further optional active ingredient-containing or active ingredient-free layer of the TTS may lastly contain additional materials that are conventional for transdermal therapeutic systems. Above all, polymers that can be cited here are those that are used in the production of transdermal systems and are physiologically safe, such as styrene-butadiene co-polymers and/or styrene-butadiene-styrene co-polymers, polybutenes or polyisobutylenes, homo- and co-polymers of (meth)acrylates, polyvinyl ethers, polyisoprene rubbers, and silicones. Of the (meth)acrylate co-polymers, for example the co-polymers of alkyl acrylates and/or alkyl methacrylates and further unsaturated monomers can be cited, such as acrylic acid, methacrylic acid, acrylamide, dimethylacrylamide, dimethylamino ethyl acrylamide, acrylonitrile and/or vinyl acetate.

In accordance with a particularly preferred embodiment, the at least one dopamine agonist-containing matrix based on polybutylene, in particular based on polyisobutylene, comprises a further polymer which is selected from the class of styrene-butadiene (SB) co-polymers and/or the styrene-butadiene-styrene (SBS) co-polymers. Such an SB or SBS co-polymer may be procured for example from the company Henkel (Germany).

In accordance with an advantageous embodiment, the co-polymer from the class of styrene-butadiene and/or styrene-butadiene-styrene has adhesive properties which advantageously ensure a good bonding of the at least one dopamine agonist-containing matrix with the rear layer of the transdermal system, such that, in particular under the forces of gravity, which may occur during the period of application of the transdermal system to the skin, or also in the event of an ingress of moisture, for example as a result of shower water and/or perspiration water, a robust bond between rear layer and active ingredient-containing layer may still be ensured, but without negatively influencing the adhesive properties of the transdermal system on the skin.

In accordance with a very particularly preferred embodiment, the at least one dopamine agonist-containing matrix based on polybutylene, in particular based in polyisobutylene, comprises at least one styrene-butadiene and/or styrene-butadiene-styrene co-polymer, wherein the dopamine agonist is selected from a dopamine D₂ agonist, preferably from quinagolide, rotigotine, bromocriptine, cabergoline, apomorphine, pramipexole, ropinirole, propylnorapomorphine, sumanirole, pergolide, piribedile, dihydroergocryptine and/or lisuride, in particular from rotigotine.

Insofar as a particularly preferred embodiment comprises a polymer from the class of styrene-butadiene and/or styrene-butadiene-styrene co-polymers, a preferred ratio of the at least one polybutylene or polyisobutylene and of the at least one styrene-butadiene and/or styrene-butadiene-styrene co-polymers is at least approximately 1 to approximately 1, particularly preferably at least approximately 2 to approximately 1, in particular approximately 2.3 to approximately 1. At most, a preferred ratio of the at least one polybutylene or polyisobutylene and of the at least one styrene-butadiene and/or styrene-butadiene-styrene co-polymers is up to approximately 6 to approximately 1, particularly preferably up to approximately 5 to approximately 1, in particular up to approximately 4 to approximately 1. The ratio relates here to the total content (% by weight) or one or more polymers from the class of polybutylenes or polyisobutylenes to the total content (% by weight) of one or more polymers from the class of styrene-butadiene and/or styrene-butadiene-styrene co-polymers in the dried matrix.

In principle, the molecular weight of the at least one polybutylene of a TDS according to the invention comprising at least one dopamine agonist-containing matrix may very within a wide range. The molecular weight of the at least one polybutylene or polyisobutylene, however, preferably is at least approximately 20,000 g/mol, particularly preferably at least approximately 45,000 g/mol, in particular at least approximately 60,000 g/mol. The molecular weight of the at least one polybutylene or polyisobutylene preferably is up to approximately 100,000 g/mol, particularly preferably up to approximately 85,000 g/mol, in particular up to approximately 75,000 g/mol. A polybutylene polymer or polyisobutylene polymer having these properties will also be referred to hereinafter as a “medium-molecular polybutylene polymer”.

In accordance with a preferred embodiment comprises the at least one matrix based on polybutylene, in particular based on polyisobutylene, but at least two polymers from the class of polybutylenes or polyisobutylenes. A first of the at least two polybutylenes is particularly preferably a medium-molecular polybutylene polymer or polyisobutylene polymer.

Insofar as a second polymer based on polybutylene is contained in the at least one matrix, this second polybutylene or polyisobutylene preferably has a molecular weight of at least approximately 500,000 g/mol, in particular of at least approximately 800,000 g/mol. At most, a preferred molecular weight of a second polymer based on polybutylene or polyisobutylene is up to approximately 3,500,000 g/mol, in particular up to approximately 1,200,000 g/mol. A polybutylene polymer or polyisobutylene polymer having these properties will also be referred to hereinafter as a “high-molecular polybutylene polymer”.

In accordance with a particularly preferred embodiment, a first polybutylene or polyisobutylene polymer of an advantageous TDS has a preferred molecular weight of at least approximately 60,000 g/mol and/or of at most approximately 75,000 g/mol and/or a second polybutylene or polyisobutylene has a preferred molecular weight of at least approximately 800,000 g/mol and/or of at most approximately 1,200,000 g/mol.

A ratio of the at least two polybutylene or polyisobutylene polymers may vary in principle within a wide range. Hereinafter, the ratio of the at least two polybutylene or polyisobutylene polymers relates to the ratio of the proportion (% by weight) of a first polybutylene or polyisobutylene polymer to the proportion (% by weight) of a second polybutylene or polyisobutylene polymer in the dried matrix. The ratio of a first polybutylene or polyisobutylene polymer and of a second polybutylene or polyisobutylene polymer, however, is at most approximately 9 to approximately 0.1, particularly preferably at most approximately 7 to approximately 0.5, in particular at most approximately 6 to approximately 1.

Lastly, an active ingredient-containing matrix may also comprise more than two polybutylenes, for example three polybutylenes.

The matrix of a TDS may additionally contain one or more solubility enhancers which advantageously enable a good solubility of the dopamine agonists in the matrix. Solubility enhancers may be selected here from the group of hydrophilic polymers, such as polyvinyl pyrrolidone (PVP), co-polymers of vinyl pyrrolidone and vinyl acetate, polyethylene glycol, polypropylene glycol, co-polymers of ethylene and vinyl acetate, and also glycerol and esters thereof. Particularly preferred solubility enhancers comprise a vinyl pyrrolidone-vinyl acetate co-polymer and/or PVP and may be procured for example under the name Kollidon VA 64 or Kollidon CL-M from the company BASF (Germany).

The content of a solubility enhancer, preferably of a vinyl pyrrolidone-vinyl acetate co-polymer and/or of a PVP, in the advantageous TDS lies preferably in the range of from 1 to 10% by weight, particularly preferably in the range of from 1.5 to 8% by weight.

The content of a vinyl pyrrolidone-vinyl acetate co-polymer preferably is at least approximately 4% by weight, in particular preferably approximately 6% by weight. At most, the content of a vinyl pyrrolidone-vinyl acetate co-polymer is up to approximately 8% by weight. The content of a PVP is preferably at least approximately 1% by weight, in particular preferably approximately 2% by weight. At most, the content of a PVP is preferably up to approximately 4% by weight, in particular up to approximately 3% by weight.

It has been found that the delivery of the at least one dopamine agonist to the skin or into the skin may improve if an absorption enhancer from the group of isopropyl esters, for example from esterifications of propanol with carboxylic acids consisting of 8-20 carbon atoms, is part of the matrix of an advantageous TDS. Isopropyl myristate is suitable as a particularly preferred absorption enhancer and can be procured for example from the company Merck (Germany).

The content of an absorption enhancer, in particular of isopropyl myristate, in the advantageous TDS is preferably at least approximately 4% by weight, particularly preferably at least approximately 6% by weight, in particular at least approximately 7% by weight, in particular preferably at least approximately 7.8% by weight. At most, a preferred content of an absorption enhancer, in particular of isopropyl myristate, is up to approximately 12% by weight, particularly preferably up to approximately 10% by weight, in particular up to approximately 9% by weight, in particular preferably up to approximately 8.2% by weight.

The delivery rate of the dopamine agonist may advantageously be increased by adding an optional cosolvent which is selected from the group of aliphatic alcohols. Such an aliphatic alcohol is preferably selected from the group of linear aliphatic alcohols, preferably with a chain length between 10 and 14 carbon atoms. The number of PH groups may vary in principle between 1 and the number of carbon atoms. A preferred aliphatic alcohol in this case is n-dodecanol, however 1-dodecanol is particularly preferred and may be procured for example under the name Selectophore™ from the company Sigma-Aldrich (Germany).

The content of the aliphatic alcohol, preferably of n-dodecanol, in particular of 1-dodecanol, in the advantageous TDS is preferably at least approximately 4% by weight, particularly preferably at least approximately 6% by weight, in particular at least approximately 7% by weight, in particular preferably at least approximately 7.8% by weight. At most, a preferred content of an aliphatic alcohol, in particular of n-dodecanol, is up to approximately 12% by weight, particularly preferably up to approximately 10% by weight, in particular up to approximately 9% by weight, in particular preferably up to approximately 8.2% by weight.

A content of one or more of the preferred cosolvent(s) and/or absorption enhancer(s) advantageously results in a controlled and sustained release of active ingredient from the preferred transdermal therapeutic system, particularly preferably from a transdermal therapeutic system comprising a dopamine agonist from the class of D₂ agonist and the ergot alkaloid derivatives, more preferably from the class of D₂ agonists, in particular from rotigotine, pramipexole and/or ropinirole, in particular preferably from rotigotine.

An advantageous composition of a dopamine agonist-containing matrix based on polybutylene, in particular based on polyisobutylene, comprises
(i) 0.3 to 50% by weight of a D₂ agonist,
(ii) 15 to 80% by weight of the medium molecular or high molecular polybutylene, preferably of the medium molecular polybutylene or polyisobutylene,
(iii) 2 to 10% by weight of vinyl pyrrolidone-vinyl acetate co-polymer,
(iv) optionally from 4 to 12% by weight isopropyl myristate, and
(v) optionally from 4 to 12% by weight n-dodecanol.

One specific composition of a dopamine agonist-containing matrix based on polybutylene preferably comprises
(i) 3 to 15% by weight rotigotine, in particular 4.5 to 12.5% by weight rotigotine,
(ii) 3 to 65% by weight of the medium molecular polybutylene or polyisobutylene,
(iii) 5 to 35% by weight of the high molecular polybutylene or polyisobutylene,
(iv) 4 to 8% by weight of vinyl pyrrolidone-vinyl acetate co-polymer,
(v) 6 to 10% by weight isopropyl myristate, and
(vi) optionally from 6 to 10% by weight 1-dodecanol.

The constituents of a dopamine agonist-containing matrix based on polybutylene are particularly preferably selected from
(i) 6 to 10% by weight rotigotine,
(ii) 35 to 63% by weight of the medium molecular polybutylene or polyisobutylene,
(iii) 5 to 11% by weight of the high molecular polybutylene or polyisobutylene,
(iv) 5 to 8% by weight of vinyl pyrrolidone-vinyl acetate co-polymer,
(v) 1 to 3% by weight PVP,
(vi) 7 to 9% by weight isopropyl myristate, and
(vii) 7 to 9% by weight 1-dodecanol.

In particular, the constituents of a dopamine agonist-containing matrix based on polybutylene are selected from
(i) 6 to 10% by weight rotigotine,
(ii) 35 to 63% by weight of the medium molecular polybutylene or polyisobutylene,
(ii) 5 to 11% by weight of the high molecular polybutylene or polyisobutylene,
(iii) 10 to 25% by weight of an SB and/or SBS co-polymer,
(iv) 5 to 8% by weight of vinyl pyrrolidone-vinyl acetate co-polymer,
(v) 1 to 4% by weight PVP,
(vi) 7 to 9% by weight isopropyl myristate, and
(vii) 7 to 9% by weight 1-dodecanol.

In particular, the constituents of a dopamine agonist-containing matrix based on polybutylene are preferably selected from
(i) 6 to 10% by weight rotigotine,
(ii) 35 to 63% by weight of the medium molecular polybutylene or polyisobutylene,
(ii) 5 to 11% by weight of the high molecular polybutylene or polyisobutylene,
(iii) 15 to 25% by weight of an SB and/or SBS co-polymer,
(iv) 5 to 8% by weight of vinyl pyrrolidone-vinyl acetate co-polymer,
(v) 1 to 3% by weight PVP,
(vi) 7 to 9% by weight isopropyl myristate, and
(vii) 7 to 9% by weight 1-dodecanol.

In particular, a preferred content of rotigotine is at least approximately 7 up to at most approximately 9% by weight, a content of a first polybutylene polymer or polyisobutylene polymer is preferably up to approximately 60% by weight, in particular approximately 57% by weight, and a preferred content of a second polybutylene polymer or polyisobutylene polymer is at least approximately 8% by weight, in particular approximately 10% by weight. The first polybutylene or polyisobutylene polymer particularly preferably has a molecular weight in the range of from approximately 60,000 g/mol to approximately 75,000 g/mol, and the second polybutylene or polyisobutylene polymer has a molecular weight in the range of from approximately 800,000 g/mol to approximately 1,200,000 g/mol.

Insofar as a preferred embodiment of an advantageous TDS comprises an SB and/or SBS co-polymer, a preferred content of a first polybutylene polymer or polyisobutylene polymer is preferably up to approximately 45% by weight, in particular up to approximately 40% by weight, and a preferred content of a second polybutylene polymer or polyisobutylene polymer is at least approximately 5% by weight, in particular approximately 7% by weight. The preferred content of an SB and/or SBS co-polymer is at least approximately 10% by weight, preferably at least approximately 15% by weight, in particular approximately 20% by weight. At most, a preferred content of an SB and/or SBS co-polymer is up to approximately 30% by weight, particularly preferably up to approximately 25% by weight.

TDS may also comprise a control membrane. If TDS that comprise only one dopamine agonist-containing matrix layer, the control membrane may be laminated onto the application-side matrix. In order to achieve the adhesive properties of the TDS, an application-side dopamine agonist-containing or active ingredient-free matrix is in this case additionally applied to the control membrane.

In particular, a control membrane is based on a polymer selected from the group of polyolefins, olefin-co-polymers, polyesters, co-polyesters, polyamides, co-polyamides, polyurethanes and the like. Polyesters and, of these, in particular polyethylene terephthalates and polycarbonates, polyolefins such as polyethylenes, polypropylenes or polybutylenes, polyethylene oxides, polyurethanes, polystyrenes, polyamides, polyimides, polyvinyl acetates, polyvinyl chlorides, polyvinylidene chlorides, co-polymers such as acrylonitrile-butadiene-styrene terpolymers, or ethylene-vinyl acetate co-polymers may be cited as examples of suitable materials. A preferred control membrane comprises a polypropylene and may be procured for example under the name Celgard from the company Azelis (Germany).

Insofar as the TDS according to the invention comprises more than one matrix, the weight per unit area of a first or further matrix moves particularly preferably at least at approximately 15 mg/10 cm², in particular at least at approximately 30 mg/10 cm², in particular preferably at least at approximately 40 mg/10 cm². At most, a particularly preferred weight per unit area of a first or further matrix is up to approximately 70 mg/10 cm², in particular up to approximately 60 mg/10 cm², in particular preferably up to approximately 50 mg/10 cm².

However, a first or further matrix does not have to have the same weight per unit area, rather a first matrix for example may have a weight per unit area of at least approximately 30 mg/10 cm² and/or at most approximately 50 mg/10 cm², and a further matrix may have a weight per unit area of for example at least approximately 10 mg/10 cm² and/or of at most approximately 35 mg/10 cm². Insofar as an advantageous transdermal therapeutic system for an application period of approximately a day is provided, a preferred weight per unit area of a first matrix is at least approximately 10 mg/10 cm², in particular approximately 15 mg/10 cm². A weight per unit area of a second matrix is at most approximately 45 mg/10 cm², in particular approximately 35 mg/10 cm².

A coordination of the weights per unit area with the total layer thickness or the total weight per unit area of the TDS according to the invention appears to be expedient particularly if a first initial delivery of an active ingredient from an application layer having an active ingredient content is to be coordinated with a second longer-lasting active ingredient delivery from a matrix layer facing the rear layer. Likewise, the influence on the wearing properties of the TDS must be taken into consideration when choosing the weight per unit area.

In order to prevent a migration of active ingredient from the matrix based on polybutylene, a preferred transdermal delivery system advantageously comprises an occlusive rear layer. What are known as backing films, for example consisting of polyester with a thickness of preferably at least approximately 5 μm, particularly preferably of at least approximately 7.5 μm, in particular of at least approximately 10 μm, in particular preferably at least approximately 12 μm, are usually used as rear layers of a TDS according to the invention. At most, a backing film of this kind has a preferred thickness of up to approximately 200 μm, particularly preferably of up to approximately 150 μm, in particular of up to approximately 100 μm, in particular preferably of up to approximately 50 μm, most preferably of up to approximately 30 μm. Such backing films are flexible and may lie optionally around the edges of the matrix layer, i.e. around the side faces of the active ingredient-containing matrix pointing in lateral directions, and may cover same.

A rear layer, in particular an occlusive rear layer, is preferably based on a polymer selected from the group consisting of polyolefins, olefin co-polymers, polyesters, co-polyesters, polyamides, co-polyamides, polyurethanes and the like. Polyesters and, of these, in particular polyethylene terephthalates (PET) and polycarbonates, polyolefins such as polyethylenes, polypropylenes or polybutylenes, polyethylene oxides, polyurethanes, polystyrenes, polyamides, polyimides, polyvinyl acetates, polyvinyl chlorides, polyvinylidene chlorides, co-polymers such as acrylonitrile-butadiene-styrene terpolymers, or ethylene-vinyl acetate co-polymers may be cited as examples of suitable materials. A preferred material of a rear layer is selected from a polyester, particularly preferably from a polyethylene terephthalate. A rear layer of this kind may be procured for example under the name Hostaphan MN 15 DMF from the company Mitsubishi Polyester Film GmbH (Germany).

A rear layer of a TDS according to the invention may also comprise a cover layer or an overtape, which protrudes laterally beyond the edges of the at least one dopamine agonist-containing matrix based on polybutylene and thus may enable an improved adhesion of the TDS according to the invention to the skin. A preferred cover layer or a preferred overtape is occlusive in this case.

The overtape may also be multi-layered. In particular, an overtape comprises an active ingredient-free adhesive layer and an overtape film, wherein an overtape film preferably comprises a polymer selected from the group of polyolefins, olefin co-polymers, polyesters, co-polyesters, polyamides, co-polyamides, polyurethanes and the like. Polyesters and, of these, in particular polyethylene terephthalates and polycarbonates, polyolefins such as polyethylenes, polypropylenes or polybutylenes, polyethylene oxides, polyurethanes, polystyrenes, polyamides, polyimides, polyvinyl acetates, polyvinyl chlorides, polyvinylidene chlorides, co-polymers such as acrylonitrile-butadiene-styrene terpolymers, or ethylene-vinyl acetate co-polymers may be cited as examples of suitable materials. A preferred material of an overtape is selected from a polyester, particularly preferably from a polyethylene terephthalate.

In one specific embodiment of the TTS according to the invention, as mentioned above, the dopamine agonist-containing matrix based on polybutylene or a dopamine agonist-containing or active ingredient-free application layer may be covered by a removable protective film also referred to in technical terminology as a release liner. When storing the TTS, the active ingredient-containing matrix or the application layer is hereby effectively protected against mechanical influences and/or an undesirable ingress of air. Due to the suppression of an undesired ingress of air, a breakdown of dopamine agonists contained in the matrix, in particular of oxygen-sensitive dopamine agonists is prevented, and the storage or long-term stability of the patch is thus improved. For application of the patch, the protective film of the patch is then firstly removed, prior to fastening the system to the skin. For improved grip and to thereby facilitate the removal of the protective film, the protective film in the case of advantageous patches protrudes beyond the edge of the rest of the patch.

Embodiments according to the invention of a transdermal therapeutic system, as mentioned, comprise an active ingredient matrix containing at least one dopamine agonist based on polybutylene or polyisobutylene. In accordance with a preferred embodiment the dopamine agonist disperses to be dissolved in the matrix forming polymer or polymer blend based on polybutylene or based on polyisobutylene.

In accordance with one specific solution in the sense of the present invention, a mixture of a solvent and of a solvate is understood here, wherein the solvate may be molecularly disperse, that is to say may have a particle size of less than 1 nm. Furthermore, a solution may also comprise colloidally dispersely dissolved particles with a size in the range of from 1 nm to 1 μm and/or roughly dispersely dissolved particles with a size of more than 1 μm.

In accordance with the method according to the invention, the above-mentioned constituents required for the production of the dopamine agonist-containing matrix are therefore advantageously stirred into a suitable solvent. The coating compound produced in this way is coated on a release liner and is dried in order to remove the used solvent(s). Insofar as a matrix of a particularly preferred embodiment comprises one or more of the above-mentioned SB and/or SBS co-polymers, at least one of the co-polymers is admixed as further constituent to obtain a coating compound.

A drying as explained above may be performed for example at a preferred temperature of at least approximately 40° C., particularly preferably of at least approximately 60° C., in particular of at least approximately 70° C. The drying may preferably take place over a period of time of up to several hours, particularly preferably over a period of time of up to approximately 30 minutes, in particular over a period of time of up to approximately 15 minutes, in particular preferably over a period of time of up to approximately 5 minutes, for example of from approximately 1 to approximately 3 or approximately 4 or approximately 5 minutes. At most, however, a preferred temperature is up to approximately 130° C., particularly preferably up to approximately 100° C., in particular up to approximately 95° C.

A drying or also a temperature control of the active ingredient-containing matrix and/or of the laminate and/or the transdermal delivery systems and/or the packaged transdermal delivery systems advantageously occurs over a preferred period of time of at least a few minutes, particularly preferably of at least approximately 30 minutes, in particular of at least approximately 8 hours. At most, a drying or temperature control occurs for a preferred period of time of up to a week, particularly preferably of up to three days, in particular of up to 48 hours, in particular preferably of approximately 24 hours. Here, temperature control is understood to mean a heating of the active ingredient-containing matrix and/or of the laminate and/or the punched-out transdermal therapeutic systems and/or the packaged transdermal therapeutic systems to a moderately warm temperature well suited to the requirements, at which temperature the active ingredient-containing matrix or the active ingredient itself does not change disadvantageously, for example does not undergo a significant physicochemical change and/or does not experience any crystallisation of the active ingredient.

Such an approach advantageously enables a complete dissolution or the dopamine agonist(s), in particular of rotigotine, and prevents a formation of crystal nuclei, whereby a recrystallisation of the active ingredient may be prevented or at least delayed. Furthermore, the water content of an active ingredient-containing matrix based on polybutylene or polyisobutylene dried in this way may be adjusted to a lowest possible value. Preferred TDS therefore have a water content of up to approximately 2% by weight, preferably of up to approximately 1% by weight, particularly preferably of up to approximately 0.75% by weight, in particular of up to approximately 0.5% by weight, in particular preferably of up to approximately 0.2% by weight.

In addition, a preferred method according to the invention may consider the use of a pre-dried packing or packaging material, in particular a bag, in which the TDS are packaged and which may also be provided with moisture-absorbing materials.

A preferred packaged transdermal delivery system may thus contain a desiccant which is included loosely or in fixed fashion in the packaging, preferably in a bag, moreover a desiccant particularly preferably based on a thermoplastic, in particular based on polyethylene. Such a desiccant or moisture-absorbing material is obtainable for example under the name Activ-Film™ from the company CSP Technologies and may be introduced into the bag in a packaging step, whereby the moisture content, in particular the water content, in the ambient environment of the packaged TDS during storage may additionally be reduced. An active ingredient-containing matrix based on polybutylene or polyisobutylene thus has a lower content of up to approximately 2% by weight, preferably of up to approximately 1% by weight, particularly preferably of up to 0.75% by weight, in particular of up to approximately 0.5% by weight, in particular preferably of up to approximately 0.2% by weight, even after a longer storage period, whereby the recrystallisation of the active ingredient in the matrix may be prevented.

In order to keep the moisture content, in particular the water content, in the dried active ingredient-containing matrix of the TDS according to the invention as low as possible, in accordance with a preferred method a rear layer and/or a protective film having moisture-absorbing material properties, such as materials comprising a silica gel formulation, may also be used during the production of the laminate.

In accordance with a further embodiment, before or after application to a protective film, planar active ingredient cores, for example active ingredient-containing matrix cores, may be punched out from the laminate comprising an active ingredient-containing matrix based on polybutylene or based on polyisobutylene, and subsequently may be subjected to the production method according to the invention. Here, an active ingredient core is understood to mean a planar dopamine agonist-containing matrix which advantageously comprises a rear layer or a protective film on its rear side and/or on its application side and which is cut out, in particular punched out, from the above-mentioned active ingredient-containing laminate with the aid of a cutting tool, in particular a punching tool. In particular preferably, planar active ingredient cores are punched out from a laminate which comprises the active ingredient-containing matrix based on polybutylene and a protective film.

Dopamine agonists are used generally for the therapy of Parkinson's disease, amenorrhoea, acromegaly, hyperprolactinaemia, as emetics, for weaning during the breastfeeding period, and as impotence drugs. The present invention preferably comprises a transdermal therapeutic system for use in the treatment of Parkinson's disease, restless leg syndrome, amenorrhoea, acromegaly and hyperprolactinaemia. In particular Parkinson's disease and restless leg syndrome are treated particularly preferably.

Further features of the invention result from the following description of practical examples in conjunction with the claims and the figures. It should be noted that the invention is not limited to the embodiments of the described practical examples, but instead is defined by the scope of the accompanying claims. In particular, the individual features in embodiments according to the invention may be implemented in a combination other than in the examples described below. In the following explanation of a number of practical examples of the invention, reference is made to the accompanying drawings. In the drawings:

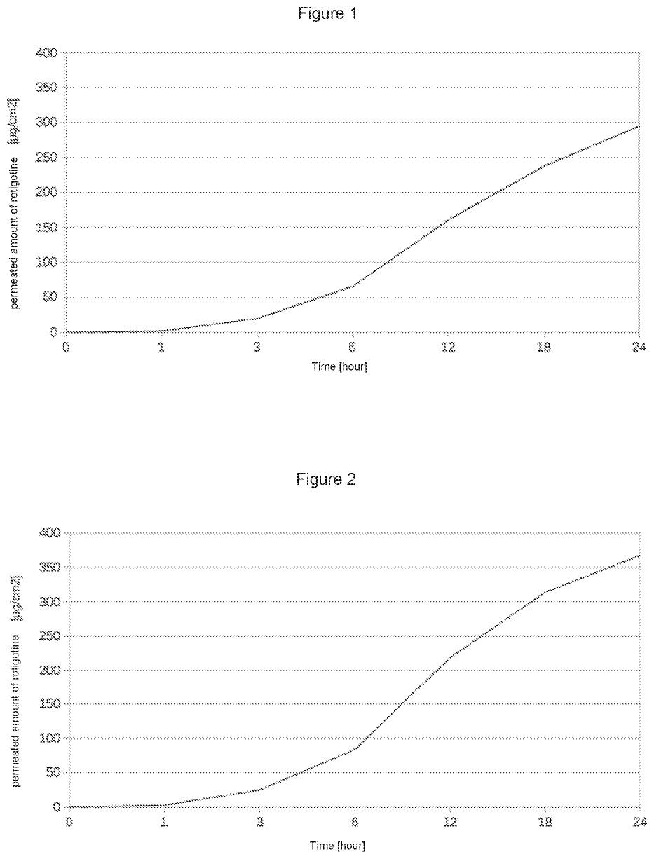

FIG. 1 shows a graph illustrating the in vitro permeation of rotigotine base (μg/cm²) from a transdermal therapeutic system according to the invention as per Example 1 through bare mouse skin. The skin permeation was performed over a period of 24 hours (h).

FIG. 2 shows a graph illustrating the in vitro permeation of rotigotine base (μg/cm²) from a transdermal therapeutic system according to the invention as per Example 2 through bare mouse skin. The skin permeation was performed over a period of 24 hours (h).

### EXAMPLES

The production of transdermal therapeutic systems in accordance with the present invention and also the permeation of the active ingredient through the skin performed by said systems will be described hereinafter.

### Example 1

Composition of the TDS according to the invention (based on the dried matrix):

| | |
|---|---|
| rotigotine base: | 9% by weight |
| PIB adhesive (DT 87-6908): | 67% by weight |
| vinyl pyrrolidone-vinyl acetate | 6% by weight |
| co-polymer (Kollidon VA 64): | |
| PVP (Kollidon CL-M): | 2% by weight |
| isopropyl myristate: | 8% by weight |
| 1-dodecanol: | 8% by weight |

The solubility enhancer (the vinyl pyrrolidone-vinyl acetate co-polymer (Kollidon VA 64, BASF Germany, Ludwigshafen)) was dissolved in ethanol, and the polyvinyl pyrrolidone (Kollidon CL-M, BTC Europe GmbH, Germany) was stirred in. Rotigotine (Olon, Italy) was dissolved in tetrahydrofuran (THF, VWR, Germany), and isopropyl myristate (AppliChem, Germany) and 1-dodecanol (Sasol, Germany) were added and then homogenised. PIB adhesive (DT 87-6908, Henkel, Germany, mixing ratio of medium- to high-molecular: 85% by weight to 15% by weight) and THF were placed in a further vessel, and the previously prepared mixture was added and homogenised. The coating compound was then applied to a PE film siliconised on one side (Primeliner PET 75 μm 15, Loparex, Netherlands), dried at 60° C. for 15 min., and covered with a PETP film (Hostaphan MN 15 DMF, Mitsubishi Polyester Films, Germany). The resultant weight per unit area in the dried state was 50 mg/10 cm².

### Example 2

Composition of the TDS according to the invention (based on the dried matrix):

| | | |
|---|---|---|
| rotigotine base: | 9% | by weight |
| PIB adhesive (DT 87-6908): | 46.9% | by weight |
| SB adhesive (DT 87-6911): | 20.1% | by weight |
| vinyl pyrrolidone-vinyl acetate | 6% | by weight |
| co-polymer (Kollidon VA 64): | | |
| PVP (Kollidon CL-M): | 2% | by weight |
| isopropyl myristate: | 8% | by weight |
| 1-dodecanol: | 8% | by weight |

The solubility enhancer, (the vinyl pyrrolidone-vinyl acetate co-polymer (Kollidon VA 64, BASF Germany, Ludwigshafen)) was dissolved in ethanol, and the polyvinyl pyrrolidone (Kollidon CL-M, BTC Europe GmbH, Germany) was stirred in. Rotigotine (Olon, Italy) was dissolved in tetrahydrofuran (THF, VWR, Germany), and isopropyl myristate (AppliChem, Germany) and 1-dodecanol (Sasol, Germany) were added and then homogenised. PIB adhesive (DT 87-6908, Henkel, Germany, mixing ratio of medium- to high-molecular: 85% by weight to 15% by weight) and SB adhesive (DT-87-6911, Henkel, Germany) and THF were placed in a further vessel, and the previously prepared mixture was added and homogenised. The coating compound was then applied to a PE film siliconised on one side (Primeliner PET 75 μm 1S, Loparex, Netherlands), dried at 60° C. for 15 min., and covered with a PETP film (Hostaphan MN 15 DMF, Mitsubishi Polyester Films, Germany). The resultant weight per unit area in the dried state was 50 mg/10 cm².

### Execution of an In Vitro Skin Permeation of the TDS According to Examples 1 and 2:

In vitro skin permeations were performed on the basis of Franz cells, in which the donor and the acceptor chamber are separated from one another vertically by a mouse skin (bare mouse skin, Harlan Laboratories, Netherlands). For preparation of the donor side, the skin was moistened with acceptor medium (phosphate buffer 50 mM, pH 6) and applied to the opening of the Franz cell (opening area 0.98 cm²). The TDS according to Example 1 or Example 2 was punched out, in each case in a size of 0.98 cm², and applied centrally to the skin.

A magnetic stir bar was introduced into the acceptor chamber filled with acceptor medium. The cells were sealed shut and fixed on a heatable magnetic stirrer (32° C.). Samples were removed at defined points in time with the aid of an automated device and were then examined by means of HPLC in respect of their rotigotine content. The permeated amounts of rotigotine are shown in FIG. 1 and FIG. 2.

1. A transdermal delivery system comprising
at least one matrix comprising at least one dopamine agonist,
an active ingredient-impermeable rear layer, and
an optional application-side protective film,
wherein the at least one matrix containing the at least one dopamine agonist comprises at least one polybutylene polymer.

2. The transdermal delivery system according to claim 1, wherein the dopamine agonist is rotigotine.

3. The transdermal delivery system according to claim 1, wherein the matrix containing the dopamine agonist also comprises at least one polymer selected from the group consisting of styrene-butadiene co-polymers and styrene-butadiene-styrene co-polymers.

4. The transdermal delivery system according to claim 1, wherein the at least one polybutylene polymer has a molecular weight of at least approximately 20,000 g/mol and/or of and at most approximately 100,000 g/mol.

5. The transdermal delivery system according to claim 1, further comprising a second polybutylene polymer has a molecular weight of at least approximately 500,000 g/mol and at most approximately 3,500,000 g/mol.

6. The transdermal delivery system according to claim 1, wherein a first polybutylene polymer from the at least one polybutylene polymer has a molecular weight of at least approximately 60,000 g/mol and at most approximately 75,000 g/mol, and a second polybutylene has a molecular weight of at least approximately 800,000 g/mol and/or of at most approximately 1,200,000 g/mol.

7. The transdermal delivery system according to claim 1, wherein the ratio of the proportions by weight of a first polybutylene polymer from the at least one polybutylene polymer and of a second polybutylene polymer is approximately 9 to approximately 0.1.

8. The transdermal delivery system according to claim 1, wherein the transdermal delivery system comprises at least one active ingredient-containing matrix, which contains at least one active ingredient.

9. The transdermal delivery system according to claim 1, wherein the at least one matrix contains a polyvinyl pyrrolidone.

10. The transdermal delivery system according to claim 1, wherein the at least one matrix contains a myristic acid isopropyl ester.

11. The transdermal delivery system according to claim 1, wherein the at least one matrix comprises 1-dodecanol.

12. The transdermal delivery system according to claim 1, wherein the at least one matrix comprises a co-polymer formed from vinyl pyrrolidone and vinyl acetate.

13. A method for producing a transdermal delivery system according to claim 1, comprising the steps:
providing at least one dopamine agonist-containing matrix containing at least one polybutylene polymer,
optionally applying the at least one dopamine agonist-containing matrix to a film in order to obtain a laminate comprising a dopamine agonist-containing matrix,
optionally drying the laminate comprising the at least one dopamine agonist-containing matrix,
optionally punching out transdermal delivery systems in order to obtain planar active ingredient cores, and
optionally packaging transdermal delivery systems.

14. The transdermal delivery system according to claim 2, wherein the transdermal delivery system is obtainable by a method comprising the steps:
providing at least one dopamine agonist-containing matrix containing at least one polymer from the class of polybutylenes, in particular polyisobutylenes,
optionally applying the at least one dopamine agonist-containing matrix to a film in order to obtain a laminate comprising a dopamine agonist-containing matrix,
optionally drying the laminate comprising the at least one dopamine agonist-containing matrix,
optionally punching out transdermal delivery systems in order to obtain planar active ingredient cores, and
optionally packaging transdermal delivery systems.

15. The transdermal delivery system according to claim 1, wherein the transdermal delivery system is packaged in a bag which contains a desiccant.

16-17. (canceled)

18. The transdermal delivery system according to claim 1, wherein the at least one polybutylene polymer is a polyisobutylene polymer.

19. The transdermal delivery system according to claim 4, wherein the at least one polybutylene polymer has a molecular weight of at least approximately 60,000 g/mol—and at most approximately 75,000 g/mol.

20. The transdermal delivery system according to claim 5, wherein the second polybutylene polymer has a molecular weight of at least approximately 800,000 g/mol and at most approximately 1,200,000 g/mol.

21. The transdermal delivery system according to claim 7, wherein the ratio of the proportions by weight of the first polybutylene polymer from the at least one polybutylene polymer and of the second polybutylene polymer is approximately 7 to approximately 0.5, in particular up to approximately 6 to approximately 1.

22. The transdermal delivery system according to claim 8, wherein the transdermal delivery system comprises at least two active ingredient-containing matricies.
